(19) United States
(12) Patent Application Publication
(10) Pub. No.: US 2018/0344514 A1
(43) Pub. Date: Dec. 6, 2018

(54) HEAT AND AROMATHERAPY BAG

(71) Applicant: Laura Sylvester, Russellville, KY (US)

(72) Inventor: Laura Sylvester, Russellville, KY (US)

(21) Appl. No.: 15/612,317

(22) Filed: Jun. 2, 2017

(51) Int. Cl. A61F 7/02; B65D 33/00; B65D 33/24; A61L 9/03; A61L 9/04; A61L 9/12

(52) U.S. Cl. CPC A61F 7/02; B65D 33/004; B65D 33/24; A61L 9/03; A61F 2007/0279; A61L 9/12; A61F 2007/0207; A61F 2007/0223; A61F 2007/0242; A61L 9/044

(57) ABSTRACT

A heat and aromatherapy bag that provides consumers with a reliable way to deliver constant, slow, dissipating heat with an infusion of essential oils. The heat and aromatherapy bag includes a repurposed sack having a front facing and an interior, a functional indicia disposed on the front facing of the repurposed sack, a plurality of rice contained within the interior of the repurposed sack and a VELCRO® fastener disposed on each side of a neck opening disposed on top of the repurposed feed sack. The repurposed sack is a repurposed antique feed sack to provide an aesthetic rustic appearance and the functional indicia indicate one of several different intangible assets that someone might be interested in cultivating or possessing. The heat and aromatherapy bag also includes a method for utilizing a heat and aromatherapy bag.

### BACKGROUND OF THE INVENTION

### Field of the Invention

The present invention is a bag. More specifically, the present invention is a heat and aromatherapy bag.

### Description of the Related Art

Currently, there are no comparable rice bags on the market that may be placed in a microwave oven for up to approximately three minutes and are able to release consistent, comfortable heat for up to an hour if desired. There are similar rice bags in existence, such as the ones available on a peer to peer e-commerce website or ETSY®.

What is needed is a heat and aromatherapy bag that provides consumers with a reliable way to deliver constant, slow, dissipating heat with an infusion of essential oils.

### SUMMARY OF THE INVENTION

The present invention is a bag. More specifically, the present invention is a heat and aromatherapy bag.

The heat and aromatherapy bag includes a repurposed sack having a front facing and an interior, a functional indicia disposed on the front facing of the repurposed sack, a plurality of rice contained within the interior of the repurposed sack and a hook and loop or VELCRO™ fastener disposed on each side of a neck opening disposed on top of the repurposed feed sack.

The heat and aromatherapy bag includes a method for utilizing a heat and aromatherapy bag, comprising the steps of having a repurposed feed sack that is filled-up with rice up to a predetermined quantity, heating the repurposed feed sack for a first predetermined period of time and applying the repurposed feed sack to a desired area for a second predetermined period of time.

It is an object of the present invention to provide a heat and aromatherapy bag that expresses a plurality of intangible assets.

It is an object of the present invention to provide a heat and aromatherapy bag that is made to resemble an antique feed sack and cover that is removable as well as washable for added flexibility, durability and convenience.

It is an object of the present invention to provide a heat and aromatherapy bag that may also be used as a cold pack by putting it in the freezer.

It is an object of the present invention to provide a heat and aromatherapy bag that is infused with essential oils to accompany each characteristic displayed on the bag.

It is an object of the present invention to provide a heat and aromatherapy bag that either produces heat from a microwave oven or removes heat when placed in a freezer, as rice contained within the bag provides efficiency since it remains hot or cold for extended periods of time.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a front view of a heat and aromatherapy bag, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

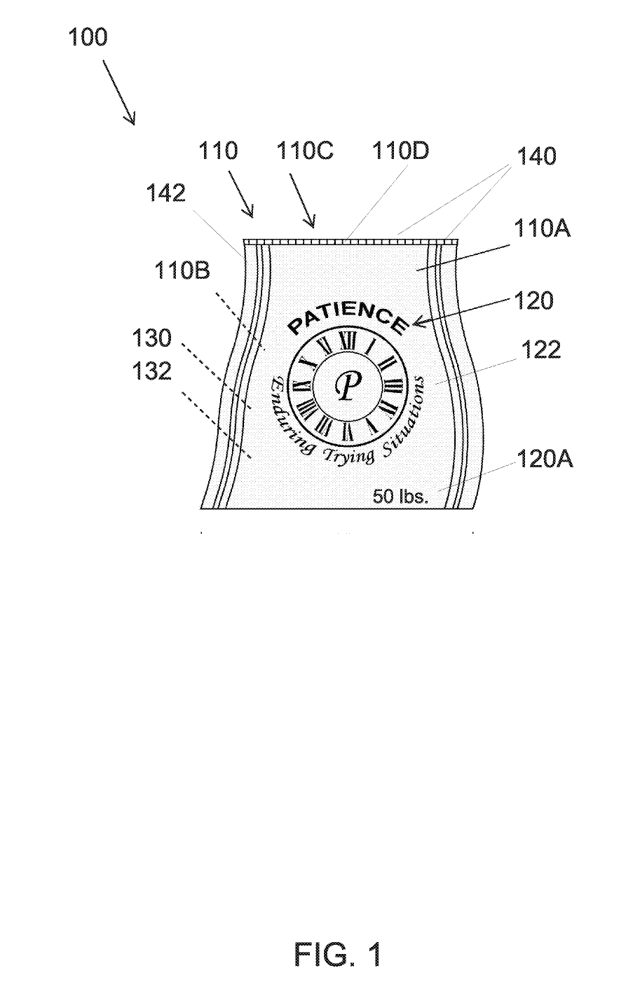

FIG. 1 illustrates a front view of a heat and aromatherapy bag 100, in accordance with one embodiment of the present invention.

The heat and aromatherapy bag 100 may include a repurposed feed sack 110, a functional indicia 120, a plurality of rice 130 and a VELCRO® fastener 140.

The repurposed feed sack 110 may have a front facing 110A and an interior 1108. The repurposed feed sack 110 may be a repurposed antique feed sack 110C to provide an aesthetic rustic appearance. The repurposed feed sack 110 may be approximately nine inches in width and ten inches in height or other suitable dimensions. The repurposed feed sack 110 may be made of heavy canvas or other suitable material. The functional indicia 120 may be disposed on the front facing 110A of the repurposed feed sack 110. The functional indicia 120 may indicate that the repurposed feed sack 110 weighs fifty pounds (although it does not actually weigh fifty pounds) to produce a mental thought of having a fifty-pound bag. The functional indicia 120 may indicate one of several different intangible assets that someone might be interested in cultivating or possessing. These intangible assets may include having enhanced or modified reality, persistence, mental toughness, ambition, success, leadership, faith, a person who prays, respect, creativity, excitement, determination, cooperation, happiness, goodwill, sowing wild oats, common sense, motivation and wedded bliss. FIG. 1 illustrates a functional indicia 120 that includes the word “PATIENCE” with a clock, a capital first letter and the additional words “enduring trying situations” 122 to facilitate inspiring patience and serenity. The rice 130 may be contained within the interior 1108 of the repurposed feed sack 110. The repurposed feed sack 110 may contain up to four cups of rice or other suitable quantity in addition to one or more essential oils 132 selected from the group consisting of lavender, peppermint, tea tree oil, patchouli and eucalyptus. The repurposed feed sack 110 may be placed in a microwave oven for approximately three minutes to deliver slow and constant heat to the repurposed feed sack 110. The rice 130 may be selected from the group consisting of white rice, brown rice, long grain rice or enriched rice. The one or more essential oils 132 may provide aromatherapy with or without heat, although typically relatively more aromatherapy is provided with heat. The VELCRO® fastener 140 may be disposed on each side of a neck opening 142 disposed on top 110D of the repurposed feed sack 110 to facilitate emptying, refilling and washing the heat and aromatherapy bag 100.

Utilization of the heat and aromatherapy bag is straightforward. The heat and aromatherapy bag may have a repurposed feed sack that is filled-up with a predetermined quantity up to four cups of rice that may be heated in a microwave oven for a predetermined period of time such as for up to three minutes and that may be applied to a desired area for up to one hour. The heat and aromatherapy bag is essential for the everyday consumer and may be readily available in all retail accessory stores, home goods stores, arts and crafts stores, drugstores, and medical equipment stores.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A heat and aromatherapy bag, comprising:
a repurposed sack having a front facing and an interior;
a functional indicia disposed on the front facing of the repurposed sack;
a plurality of rice contained within the interior of the repurposed sack; and
a Velcro fastener disposed on each side of a neck opening disposed on top of the repurposed feed sack to facilitate emptying, refilling, and washing the heat and aromatherapy bag.

2. The heat and aromatherapy bag according to claim 1, wherein the repurposed sack is a repurposed antique feed sack to provide an aesthetic rustic appearance.

3. The heat and aromatherapy bag according to claim 1, wherein the repurposed sack is nine inches in width and ten inches in height.

4. The heat and aromatherapy bag according to claim 1, wherein the functional indicia indicate one of several different intangible assets that someone might be interested in cultivating or possessing selected from the group consisting of having enhanced or modified reality, persistence, mental toughness, ambition, success, leadership, faith, a person who prays, respect, creativity, excitement, determination, cooperation, happiness, goodwill, sowing wild oats, common sense, motivation and wedded bliss.

5. The heat and aromatherapy bag according to claim 1, wherein the functional indicia indicate that the repurposed feed sack weighs fifty pounds to produce a mental thought of having a fifty-pound bag.

6. The heat and aromatherapy bag according to claim 1, wherein the repurposed sack contains up to four cups of rice.

7. The heat and aromatherapy bag according to claim 6, wherein the repurposed sack contains one or more essential oils.

8. The heat and aromatherapy bag according to claim 7, wherein the one or more essential oils provide aromatherapy with heat.

9. The heat and aromatherapy bag according to claim 7, wherein the one or more essential oils provide aromatherapy without heat.

10. The heat and aromatherapy bag according to claim 7, wherein the one or more essential oils are selected from the group consisting of lavender, peppermint, tea tree oil, patchouli and eucalyptus.

11. The heat and aromatherapy bag according to claim 1, wherein the rice is selected from the group consisting of white rice, brown rice, long grain rice or enriched rice.

12. The heat and aromatherapy bag according to claim 1, wherein the Velcro fastener is disposed on each side of a neck opening disposed on top of the repurposed feed sack to facilitate emptying, refilling, and washing the heat and aromatherapy bag.

13. A method for utilizing a heat and aromatherapy bag, comprising the steps of:
having a repurposed feed sack that is filled-up with rice up to a predetermined quantity;
heating the repurposed feed sack for a first predetermined period of time; and
applying the repurposed feed sack to a desired area for a second predetermined period of time.

14. The method for utilizing a heat and aromatherapy bag according to claim 13, wherein the predetermined quantity is filled-up with up to four cups of rice.

15. The method for utilizing a heat and aromatherapy bag according to claim 13, wherein the first predetermined period of time is heated for up to three minutes.

16. The method for utilizing a heat and aromatherapy bag according to claim 15, wherein the repurposed feed sack is heated in a microwave oven.

17. The method for utilizing a heat and aromatherapy bag according to claim 15, wherein the second predetermined period of time is applied for up to an hour.
